(19) United States
(12) Patent Application Publication
(10) Pub. No.: US 2025/0389710 A1
(43) Pub. Date: Dec. 25, 2025

(54) DETERMINING AN ACCURACY OF A MEASUREMENT OF A FLUID

(71) Applicant: Schlumberger Technology Corporation, Sugar Land, TX (US)

(72) Inventors: Anubhav Trivedi, Pune (IN); Madhuri Agrawal, Pune (IN)

(21) Appl. No.: 18/797,732

(22) Filed: Aug. 8, 2024

(30) Foreign Application Priority Data
Jun. 24, 2024 (IN) 202411048202

(51) Int. Cl. G01N 33/28; G01N 11/02; G01V 1/50

(52) U.S. Cl. CPC G01N 33/2823; G01N 11/02; G01V 1/50; G01V 2210/612; G01V 2210/74

(57) ABSTRACT

A method for measuring properties of a fluid produced from a wellbore includes receiving first data from a separator tank. The method also includes receiving second data from a flowmeter. The method also includes comparing the first data and the second data to produce compared data. The method also includes determining differences between the first data and the second data based upon the compared data.

### BACKGROUND

Fluid that is produced from a wellbore may be introduced into a separator that separates two or more portions of the fluid from one another. For example, the separator may separate an oil portion, a water portion, and a gas portion from one another. At least some of the gas portion may be bled or flared from the separator into the atmosphere. One or more properties may be measured from this atmospheric emission. These measured properties may be used to determine corresponding properties of the fluid that is produced from the wellbore.

However, as will be appreciated, there is a continued effort to reduce atmospheric emissions. Therefore, what is needed is an improved system and method to determine the properties of the fluid that is produced from the wellbore with a reduced atmospheric emission. In addition, there is a need to determine the accuracy of the measurements of this improved system and method.

### SUMMARY

A method for measuring properties of a fluid produced from a wellbore includes receiving first data from a separator tank. The method also includes receiving second data from a flowmeter. The method also includes comparing the first data and the second data to produce compared data. The method also includes determining differences between the first data and the second data based upon the compared data.

A computing system is also disclosed. The computing system includes one or more processors and a memory system. The memory system includes one or more non-transitory computer-readable media storing instructions that, when executed by at least one of the one or more processors, cause the computing system to perform operations. The operations include receiving first data from a separator tank. The separator tank separates gas from a hydrocarbon fluid that is produced from a wellbore. The first data is measured from emissions from the gas that are bled or flared from separator tank. The first data includes a plurality of properties of the hydrocarbon fluid. The properties include: a flow rate of the oil, the water, and the gas; a density of the oil, the water, and the gas; phase fractions of the oil, the water, and the gas; a line temperature of the hydrocarbon fluid; a line pressure of the hydrocarbon fluid; a differential venturi pressure of the hydrocarbon fluid; a water-to-liquid ratio (WLR) of the hydrocarbon fluid; a gas volume fraction (GVF) of the hydrocarbon fluid; or a combination thereof. The operations also include receiving second data from a flowmeter. The flowmeter measures the second data directly from the hydrocarbon fluid. The flowmeter measures the second data without separating the hydrocarbon fluid with using the separator tank. The flowmeter measures the second data without bleeding or flaring emissions from the hydrocarbon fluid. The flowmeter is an inline, surface, multi-phase flowmeter. The flowmeter includes a venturi throat. The second data is measured at a single point within the venturi throat. The second data includes the properties. The first data and the second data comprise overlapping date ranges and/or overlapping frequency ranges. The operations also include comparing the first data and the second data to produce compared data. The operations also include determining differences between the first data and the second data based upon the compared data. The operations also include generating one or more graphs to show the differences. Each of the one or more graphs shows the differences between one of the properties in the first data and the second data. The one or more graphs show the differences for a plurality of different wellbores.

A non-transitory computer-readable medium is also disclosed. The medium stores instructions that, when executed by one or more processors of a computing system, cause the computing system to perform operations. The operations include receiving first data from a separator tank. The separator tank separates oil, water, and gas from a hydrocarbon fluid that is produced from a wellbore. The first data is measured from emissions from the gas that are bled or flared from separator tank. The first data includes a plurality of properties of the hydrocarbon fluid. The properties include: a flow rate of the oil, the water, and the gas; a density of the oil, the water, and the gas; phase fractions of the oil, the water, and the gas; a line temperature of the hydrocarbon fluid; a line pressure of the hydrocarbon fluid; a differential venturi pressure of the hydrocarbon fluid; a water-to-liquid ratio (WLR) of the hydrocarbon fluid; and a gas volume fraction (GVF) of the hydrocarbon fluid. The operations also include receiving second data from a flowmeter. The flowmeter measures the second data directly from the hydrocarbon fluid. The flowmeter measures the second data without separating the hydrocarbon fluid using the separator tank. The flowmeter measures the second data without bleeding or flaring emissions from the hydrocarbon fluid. The flowmeter is an inline, surface, multi-phase flowmeter. The flowmeter includes a venturi throat. The second data is measured at a single point within the venturi throat. The second data includes the properties. The first data and the second data include overlapping date ranges and overlapping frequency ranges. The operations also include comparing the first data and the second data to produce compared data. The operations also include determining differences between the first data and the second data based upon the compared data. The operations also include generating one or more graphs to show the differences. Each of the one or more graphs shows the differences between one of the properties in the first data and the second data. The one or more graphs show the differences for a plurality of different wellbores. The operations also include identifying a first subset of the properties where the differences are less than a first predetermined threshold. The first predetermined threshold is between about 3% and about 7%. The first subset of the properties are identified via highlighting with a first color. The operations also include identifying a second subset of the properties where the differences are greater than the first predetermined threshold and less than a second predetermined threshold. The second predetermined threshold is between about 8% and about 12%. The second subset of the properties are identified via highlighting with a second color. The operations also include identifying a third subset of the properties where the differences are greater than the second predetermined threshold. The third subset of the properties are identified via highlighting with a third color.

It will be appreciated that this summary is intended merely to introduce some aspects of the present methods, systems, and media, which are more fully described and/or claimed below. Accordingly, this summary is not intended to be limiting.

### BRIEF DESCRIPTION OF THE DRAWINGS

The accompanying drawings, which are incorporated in and constitute a part of this specification, illustrate embodiments of the present teachings and together with the description, serve to explain the principles of the present teachings. In the figures:

FIG. 1 illustrates an example of a system that includes various management components to manage various aspects of a geologic environment, according to an embodiment.

FIG. 2 illustrates a schematic view of a wellsite implementing a first technique (e.g., testing bled/flared emissions from a separator) for determining properties of a fluid produced from one or more wellbores, according to an embodiment.

FIG. 3 illustrates a schematic view of the wellsite implementing a second technique (e.g., testing with an inline flowmeter) for determining properties of the fluid, according to an embodiment.

FIG. 4 illustrates a workflow for comparing the first data (measured by the separator in FIG. 2) to the second data (measured by the flowmeters in FIG. 3), according to an embodiment.

FIG. 5 illustrates a flowchart of the method for measuring properties of a fluid produced from a wellbore, according to an embodiment.

FIG. 6 illustrates a table including a sample of the first data from the separator tank, according to an embodiment.

FIG. 7 illustrates a user interface to generate the separator vs flowmeter comparison report, according to an embodiment.

FIGS. 8A and 8B illustrate a comparison report that shows both the first data (e.g., from the separator tank) and the second data (e.g., from the flowmeter(s), according to an embodiment.

FIGS. 9A-9D illustrate graphs showing trends that may be identified in the comparison report, according to an embodiment.

FIG. 10 illustrates a schematic view of a computing system for performing at least a portion of the method(s) described herein, according to an embodiment.

### DETAILED DESCRIPTION

Reference will now be made in detail to embodiments, examples of which are illustrated in the accompanying drawings and figures. In the following detailed description, numerous specific details are set forth in order to provide a thorough understanding of the invention. However, it will be apparent to one of ordinary skill in the art that the invention may be practiced without these specific details. In other instances, well-known methods, procedures, components, circuits, and networks have not been described in detail so as not to unnecessarily obscure aspects of the embodiments.

It will also be understood that, although the terms first, second, etc. may be used herein to describe various elements, these elements should not be limited by these terms. These terms are only used to distinguish one element from another. For example, a first object or step could be termed a second object or step, and, similarly, a second object or step could be termed a first object or step, without departing from the scope of the present disclosure. The first object or step, and the second object or step, are both, objects or steps, respectively, but they are not to be considered the same object or step.

The terminology used in the description herein is for the purpose of describing particular embodiments and is not intended to be limiting. As used in this description and the appended claims, the singular forms “a,” “an” and “the” are intended to include the plural forms as well, unless the context clearly indicates otherwise. It will also be understood that the term “and/or” as used herein refers to and encompasses any possible combinations of one or more of the associated listed items. It will be further understood that the terms “includes,” “including,” “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Further, as used herein, the term “if” may be construed to mean “when” or “upon” or “in response to determining” or “in response to detecting,” depending on the context.

Attention is now directed to processing procedures, methods, techniques, and workflows that are in accordance with some embodiments. Some operations in the processing procedures, methods, techniques, and workflows disclosed herein may be combined and/or the order of some operations may be changed.

### System Overview

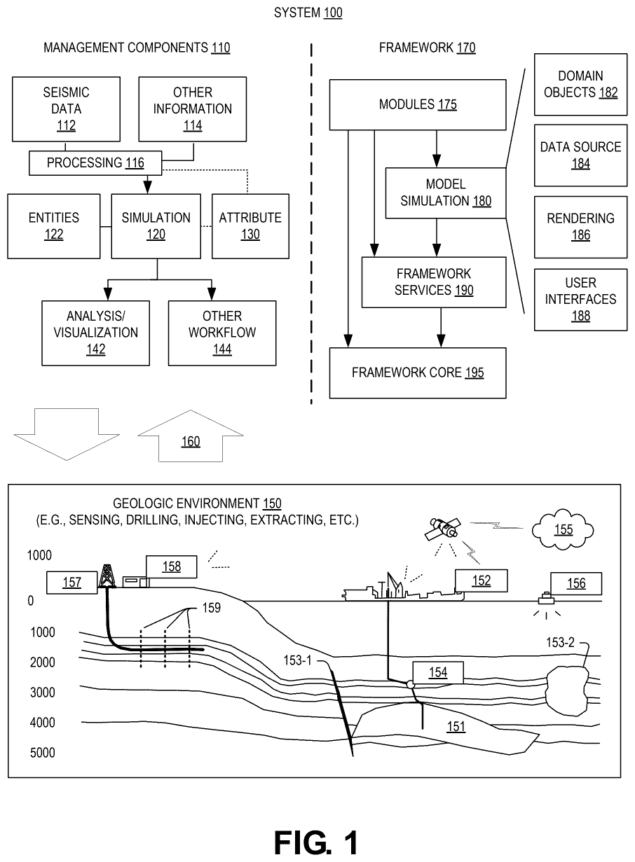

FIG. 1 illustrates an example of a system 100 that includes various management components 110 to manage various aspects of a geologic environment 150 (e.g., an environment that includes a sedimentary basin, a reservoir 151, one or more faults 153-1, one or more geobodies 153-2, etc.). For example, the management components 110 may allow for direct or indirect management of sensing, drilling, injecting, extracting, etc., with respect to the geologic environment 150. In turn, further information about the geologic environment 150 may become available as feedback 160 (e.g., optionally as input to one or more of the management components 110).

In the example of FIG. 1, the management components 110 include a seismic data component 112, an additional information component 114 (e.g., well/logging data), a processing component 116, a simulation component 120, an attribute component 130, an analysis/visualization component 142 and a workflow component 144. In operation, seismic data and other information provided per the components 112 and 114 may be input to the simulation component 120.

In an example embodiment, the simulation component 120 may rely on entities 122. Entities 122 may include earth entities or geological objects such as wells, surfaces, bodies, reservoirs, etc. In the system 100, the entities 122 can include virtual representations of actual physical entities that are reconstructed for purposes of simulation. The entities 122 may include entities based on data acquired via sensing, observation, etc. (e.g., the seismic data 112 and other information 114). An entity may be characterized by one or more properties (e.g., a geometrical pillar grid entity of an earth model may be characterized by a porosity property). Such properties may represent one or more measurements (e.g., acquired data), calculations, etc.

In an example embodiment, the simulation component 120 may operate in conjunction with a software framework such as an object-based framework. In such a framework, entities may include entities based on pre-defined classes to facilitate modeling and simulation. A commercially available example of an object-based framework is the MICROSOFT®.NET® framework (Redmond, Washington), which provides a set of extensible object classes. In the NET® framework, an object class encapsulates a module of reusable code and associated data structures. Object classes can be used to instantiate object instances for use in by a program, script, etc. For example, borehole classes may define objects for representing boreholes based on well data.

In the example of FIG. 1, the simulation component 120 may process information to conform to one or more attributes specified by the attribute component 130, which may include a library of attributes. Such processing may occur prior to input to the simulation component 120 (e.g., consider the processing component 116). As an example, the simulation component 120 may perform operations on input information based on one or more attributes specified by the attribute component 130. In an example embodiment, the simulation component 120 may construct one or more models of the geologic environment 150, which may be relied on to simulate behavior of the geologic environment 150 (e.g., responsive to one or more acts, whether natural or artificial). In the example of FIG. 1, the analysis/visualization component 142 may allow for interaction with a model or model-based results (e.g., simulation results, etc.). As an example, output from the simulation component 120 may be input to one or more other workflows, as indicated by a workflow component 144.

As an example, the simulation component 120 may include one or more features of a simulator such as the ECLIPSE™ reservoir simulator (SLB, Houston Texas), the INTERSECT™ reservoir simulator (SLB, Houston Texas), etc. As an example, a simulation component, a simulator, etc. may include features to implement one or more meshless techniques (e.g., to solve one or more equations, etc.). As an example, a reservoir or reservoirs may be simulated with respect to one or more enhanced recovery techniques (e.g., consider a thermal process such as SAGD, etc.).

In an example embodiment, the management components 110 may include features of a commercially available framework such as the PETREL® seismic to simulation software framework (SLB, Houston, Texas). The PETREL® framework provides components that allow for optimization of exploration and development operations. The PETREL® framework includes seismic to simulation software components that can output information for use in increasing reservoir performance, for example, by improving asset team productivity. Through use of such a framework, various professionals (e.g., geophysicists, geologists, and reservoir engineers) can develop collaborative workflows and integrate operations to streamline processes. Such a framework may be considered an application and may be considered a data-driven application (e.g., where data is input for purposes of modeling, simulating, etc.).

In an example embodiment, various aspects of the management components 110 may include add-ons or plug-ins that operate according to specifications of a framework environment. For example, a commercially available framework environment marketed as the OCEAN® framework environment (SLB, Houston, Texas) allows for integration of add-ons (or plug-ins) into a PETREL® framework workflow. The OCEAN® framework environment leverages.NET® tools (Microsoft Corporation, Redmond, Washington) and offers stable, user-friendly interfaces for efficient development. In an example embodiment, various components may be implemented as add-ons (or plug-ins) that conform to and operate according to specifications of a framework environment (e.g., according to application programming interface (API) specifications, etc.).

FIG. 1 also shows an example of a framework 170 that includes a model simulation layer 180 along with a framework services layer 190, a framework core layer 195 and a modules layer 175. The framework 170 may include the commercially available OCEAN® framework where the model simulation layer 180 is the commercially available PETREL® model-centric software package that hosts OCEAN® framework applications. In an example embodiment, the PETREL® software may be considered a data-driven application. The PETREL® software can include a framework for model building and visualization.

As an example, a framework may include features for implementing one or more mesh generation techniques. For example, a framework may include an input component for receipt of information from interpretation of seismic data, one or more attributes based at least in part on seismic data, log data, image data, etc. Such a framework may include a mesh generation component that processes input information, optionally in conjunction with other information, to generate a mesh.

In the example of FIG. 1, the model simulation layer 180 may provide domain objects 182, act as a data source 184, provide for rendering 186 and provide for various user interfaces 188. Rendering 186 may provide a graphical environment in which applications can display their data while the user interfaces 188 may provide a common look and feel for application user interface components.

As an example, the domain objects 182 can include entity objects, property objects and optionally other objects. Entity objects may be used to geometrically represent wells, surfaces, bodies, reservoirs, etc., while property objects may be used to provide property values as well as data versions and display parameters. For example, an entity object may represent a well where a property object provides log information as well as version information and display information (e.g., to display the well as part of a model).

In the example of FIG. 1, data may be stored in one or more data sources (or data stores, generally physical data storage devices), which may be at the same or different physical sites and accessible via one or more networks. The model simulation layer 180 may be configured to model projects. As such, a particular project may be stored where stored project information may include inputs, models, results and cases. Thus, upon completion of a modeling session, a user may store a project. At a later time, the project can be accessed and restored using the model simulation layer 180, which can recreate instances of the relevant domain objects.

In the example of FIG. 1, the geologic environment 150 may include layers (e.g., stratification) that include a reservoir 151 and one or more other features such as the fault 153-1, the geobody 153-2, etc. As an example, the geologic environment 150 may be outfitted with any of a variety of sensors, detectors, actuators, etc. For example, equipment 152 may include communication circuitry to receive and to transmit information with respect to one or more networks 155. Such information may include information associated with downhole equipment 154, which may be equipment to acquire information, to assist with resource recovery, etc. Other equipment 156 may be located remote from a well site and include sensing, detecting, emitting or other circuitry. Such equipment may include storage and communication circuitry to store and to communicate data, instructions, etc. As an example, one or more satellites may be provided for purposes of communications, data acquisition, etc. For example, FIG. 1 shows a satellite in communication with the network 155 that may be configured for communications, noting that the satellite may additionally or instead include circuitry for imagery (e.g., spatial, spectral, temporal, radiometric, etc.).

FIG. 1 also shows the geologic environment 150 as optionally including equipment 157 and 158 associated with a well that includes a substantially horizontal portion that may intersect with one or more fractures 159. For example, consider a well in a shale formation that may include natural fractures, artificial fractures (e.g., hydraulic fractures) or a combination of natural and artificial fractures. As an example, a well may be drilled for a reservoir that is laterally extensive. In such an example, lateral variations in properties, stresses, etc. may exist where an assessment of such variations may assist with planning, operations, etc. to develop a laterally extensive reservoir (e.g., via fracturing, injecting, extracting, etc.). As an example, the equipment 157 and/or 158 may include components, a system, systems, etc. for fracturing, seismic sensing, analysis of seismic data, assessment of one or more fractures, etc.

As mentioned, the system 100 may be used to perform one or more workflows. A workflow may be a process that includes a number of worksteps. A workstep may operate on data, for example, to create new data, to update existing data, etc. As an example, a may operate on one or more inputs and create one or more results, for example, based on one or more algorithms. As an example, a system may include a workflow editor for creation, editing, executing, etc. of a workflow. In such an example, the workflow editor may provide for selection of one or more pre-defined worksteps, one or more customized worksteps, etc. As an example, a workflow may be a workflow implementable in the PETREL® software, for example, that operates on seismic data, seismic attribute(s), etc. As an example, a workflow may be a process implementable in the OCEAN® framework. As an example, a workflow may include one or more worksteps that access a module such as a plug-in (e.g., external executable code, etc.).

### Vx Flowmeter Trials Vs Test Separator Data Comparison—Solidifying Vx MPFM Claim to Replace Separator Tanks

The present disclosure includes a system and method that measure one or more properties of a wellbore fluid with a surface, multi-phase flowmeter (e.g., a Vx Spectra flowmeter). The system and method described herein may measure these properties without using the conventional separator and without the atmospheric emission therefrom.

More particularly, for separators that use gas (e.g., methane) bleeding to actuate control valves, replacement by a surface, multi-phase flowmeter (e.g., a Vx Spectra flowmeter) can reduce emissions up to 99.8%. The flowmeter is a component of rapid production response solutions that enable zero flaring. The flowmeter provides real-time rate information that can be used to modify (e.g., optimize) fluid processing and/or provide assurance that fluid meeting production standards is sent to the facilities.

To support this claim, field engineers conduct trials and capture data. This data may then be compared with test separator data (e.g., collected from clients). The process includes strenuous manual efforts of copying and pasting data and fixing spreadsheets to determine the final results, which are prone to errors. The present disclosure includes systems and methods that remove the human factor from this process and automate the whole workflow.

First (e.g., client separator) data may be directly imported to interpret the data. Simultaneously, second data from the flowmeter may be analysed for the same date ranges and/or frequency ranges as the client separator data. The first and/or second data may include oil, water, and/or gas cumulative for some durations. Additionally, the data can be for sand, line temperature, line pressure, differential venturi pressure, WLR (water to liquid ratio), GVF (gas volume fraction), etc. There may be a user interface to select the channels, duration, and frequency of the data.

Both the data from flowmeter and from the separator tank may be compared, and differences may be identified (e.g., shown in different colors). For example, differences between 0-5% may be highlighted in green, 5-10% in yellow, and 10+% in red. The data may also be plotted in graphs to show the differences. In one embodiment, this may include the differences for different wells. This whole report can be exported as spreadsheet or PDF. This may save the time of field engineers, as the manual effort is now handled by the automated system. Furthermore, this report solidifies the claim that flowmeters can replace separator tanks.

The test separator may be used for well, reservoir, and/or field performance monitoring. As such, it may be used to determine fluid flow rates, determine when changes in fluid flow rates and/or composition occur (e.g., indicating water breakthrough), identify mechanical integrity issues, or a combination thereof.

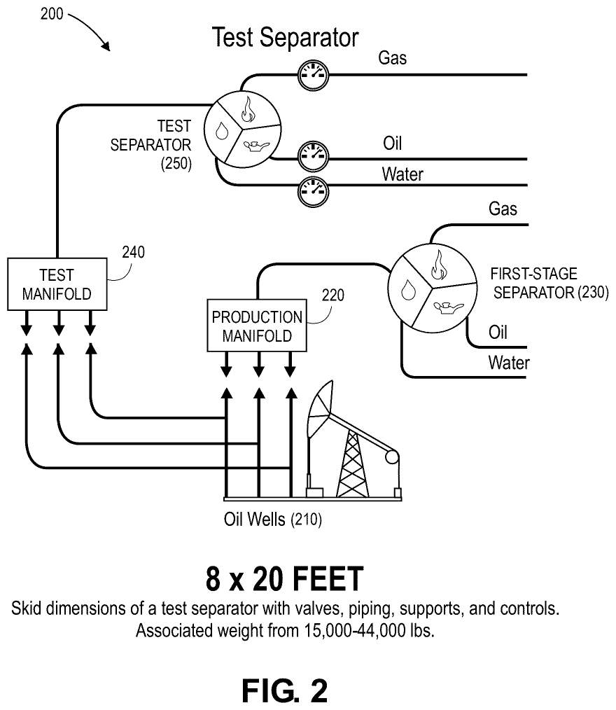

FIG. 2 illustrates a schematic view of a wellsite 200 implementing a first technique for determining properties of a fluid produced from one or more wellbores, according to an embodiment. The wellsite 200 may include one or more wells (also referred to as wellbores) 210. A hydrocarbon fluid may be produced from the wellbores 210. A first portion of the fluid may flow through a first (e.g., production) manifold 220 and then into a first (e.g., stage) separator 230. The first separator 230 may separate the first portion of the fluid into one or more portions/phases. For example, the first separator 230 may separate the first portion of the fluid into a gas portion, an oil portion, and a water portion. The gas portion, the oil portion, and/or the water portion may then be transferred downstream for further processing.

A second portion of the fluid may flow through a second (e.g., test) manifold 240 and then into a second (e.g., test) separator 250. The second separator 250 may separate the second portion of the fluid into one or more portions/phases. For example, the second separator 250 may separate the second portion of the fluid into a gas portion, an oil portion, and a water portion. The gas portion may then be bled and/or flared, which generates atmospheric emissions. First data (e.g., one or more properties) may be measured from this atmospheric emission. These measured properties may be used to determine corresponding properties of the fluid that is produced from the wellbores 210. The first data (e.g., the measured properties and/or the corresponding properties) may be or include a flow rate of the oil, the water, and the gas; a density of the oil, the water, and the gas; phase fractions of the oil, the water, and the gas; a line temperature of the hydrocarbon fluid; a line pressure of the hydrocarbon fluid; a differential venturi pressure of the hydrocarbon fluid; a water-to-liquid ratio (WLR) of the hydrocarbon fluid; a gas volume fraction (GVF) of the hydrocarbon fluid; or a combination thereof.

In an offshore production facility, the second (e.g., test) separator 250 may be considered to be a disadvantage with respect to its size and weight, particularly for high-pressure designs. It may also account for relatively high capital expenditure (CAPEX) and operating expenditures (OPEX). In addition, well measurement may take a long time (e.g., flush out previous fluids, wait for stable process conditions, etc.). The second (e.g., test) separator 250 mass means that stabilization time can be prolonged when switching wells for testing (e.g., a 12 hour well test may take 4 hours to stabilize). Additionally, the second (e.g., test) separator 250 may provide an “average” measurement of the well flowrates and is generally unable to highlight individual flow patterns.

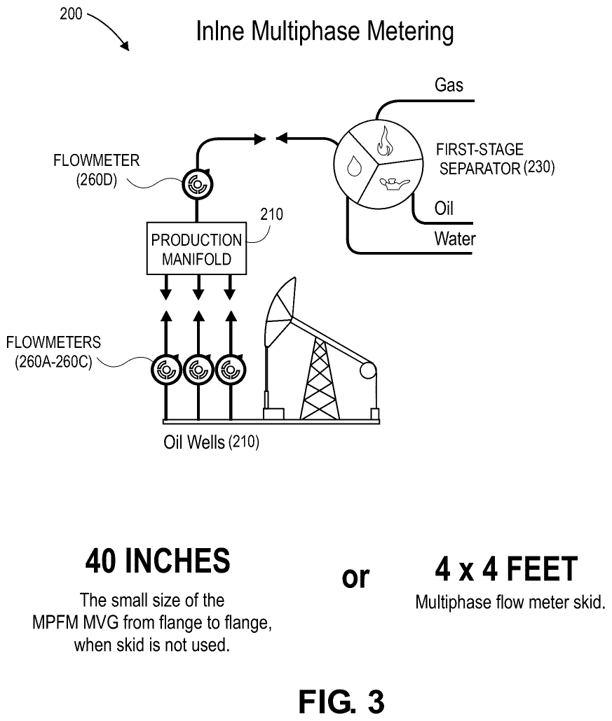

FIG. 3 illustrates a schematic view of the wellsite 200 implementing a second technique for determining properties of the fluid, according to an embodiment. In this embodiment, the second (e.g., test) manifold 240 and the second (e.g., test) separator 250 are omitted and replaced by one or more surface, inline, multi-phase flowmeters (four are shown: 260A-260D). The flowmeters 260A-260C may be positioned in lines that extend between the wellbores 210 and the first (e.g., production) manifold 220, and the flowmeter 260D may be positioned in a line that extends between the first (e.g., production) manifold 220 and the first (e.g., stage) separator 230. In other words, the flowmeters 260A-260C may be upstream from the first (e.g., production) manifold 220, and the flowmeter 260D may be downstream from the first (e.g., production) manifold 220. In an embodiment, one or more of the flowmeters 260A-260D may be placed at the wellhead to a test line, so it may be the first equipment in the flowline.

The flowmeters 260A-260D may be configured to measure second data. In one embodiment, the second data may be the same as the first data. For example, the second data may include a flow rate of the oil, the water, and the gas; a density of the oil, the water, and the gas; phase fractions of the oil, the water, and the gas; a line temperature of the hydrocarbon fluid; a line pressure of the hydrocarbon fluid; a differential venturi pressure of the hydrocarbon fluid; a water-to-liquid ratio (WLR) of the hydrocarbon fluid; a gas volume fraction (GVF) of the hydrocarbon fluid; or a combination thereof. The flowmeters 260A-260D may be configured to measure the second data without separating the hydrocarbon fluid using the second separator 250. The flowmeters 260A-260D may also or instead be configured to measure the second data without bleeding or flaring emissions from the hydrocarbon fluid. As a result, the embodiment of FIG. 3 may have lower emissions than the embodiment of FIG. 2.

The embodiment in FIG. 3 includes many advantages. More particularly, the measurements of oil, gas, and water using the flowmeters 260A-260D may provide continuous, real-time measurements, which enables changes in the well fluids to be detected earlier than using the second separator 250. Moreover, it may include fewer vessels and less piping. This enables optimized well pad design and performance of continuous flow measurements without separation. More particularly, this may enable optimized well pad design because the first level of separation may be omitted, which reduces the overall operating expenses associated with maintaining separate flowlines for oil, water, and gas. In addition, the flaring of the gas may be omitted, resulting as a process of this separation. It also removes the cost of keeping the test separator tanks. It also allows the ability to monitor more wellbores 210 rather than spending time on routine manual work. This results in greater efficiency and greater productivity.

Additional advantages of using a multiphase flowmeter (MPFM) 260A-260D instead of the second (e.g., test) separator 250 in an oil and gas field may include faster stabilization. More particularly, MPFMs can stabilize process conditions (e.g., temperature) more quickly due to their smaller mass, reducing stabilization times from hours to minutes. The MPFMs also provide shorter test durations. More particularly, faster stabilization leads to shorter overall test times, including both test time and stabilization time. The MPFMs also enable direct flow measurements, which are more representative of well flows compared to averaged or conditioned flows from the second (e.g., test) separator 250. The MPFMs also provide reduced well test durations. More particularly, they provide ability to meter direct flow measurements, and faster stabilization contributes to reducing overall well test durations when using the MPFMs. The MPFMs also provide improved accuracy in measuring flow rates, providing more precise data for analysis and decision-making. The MPFMs are also smaller than the second (e.g., test) separator 250, making them easier to install and operate in offshore production facilities. In addition, due to their smaller size and simpler installation requirements, MPFMs may incur lower installation costs compared to the second (e.g., test) separator 250.

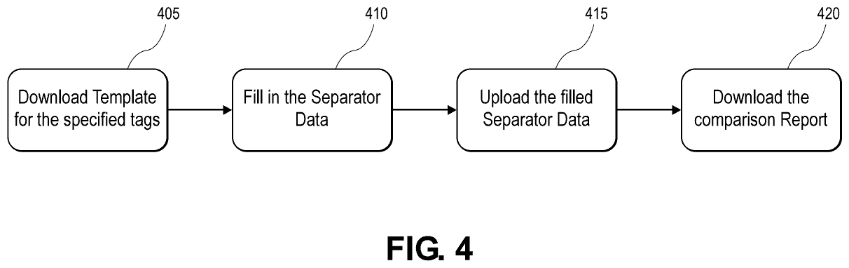

FIG. 4 illustrates a workflow for comparing the first data measured by the second (e.g., test) separator 250 in FIG. 2 to the second data measured by the flowmeters 260A-260D in FIG. 3, according to an embodiment. A template may be downloaded for the specified tags, as at 405. The second data (e.g., from the test separator 250) may be filled into the template, as at 410. Then, the template with the second data may be uploaded, as at 415. Finally, a report comparing the first data and the second data may be downloaded, as at 420.

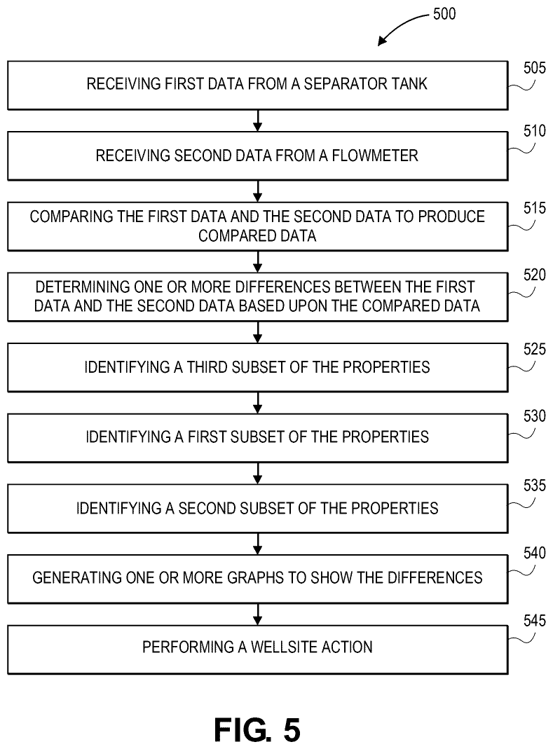

FIG. 5 illustrates a flowchart of the method 500 for measuring properties of a fluid produced from a wellbore 210, according to an embodiment. More particularly, the method 500 may determine an accuracy of a measurement of properties of a fluid produced from one or more wellbores 210. An illustrative order of the method 500 is provided below; however, one or more portions of the method 500 may be performed in a different order, simultaneously, repeated, or omitted. At least a portion of the method 500 may be performed with a computing system 1000 (described below).

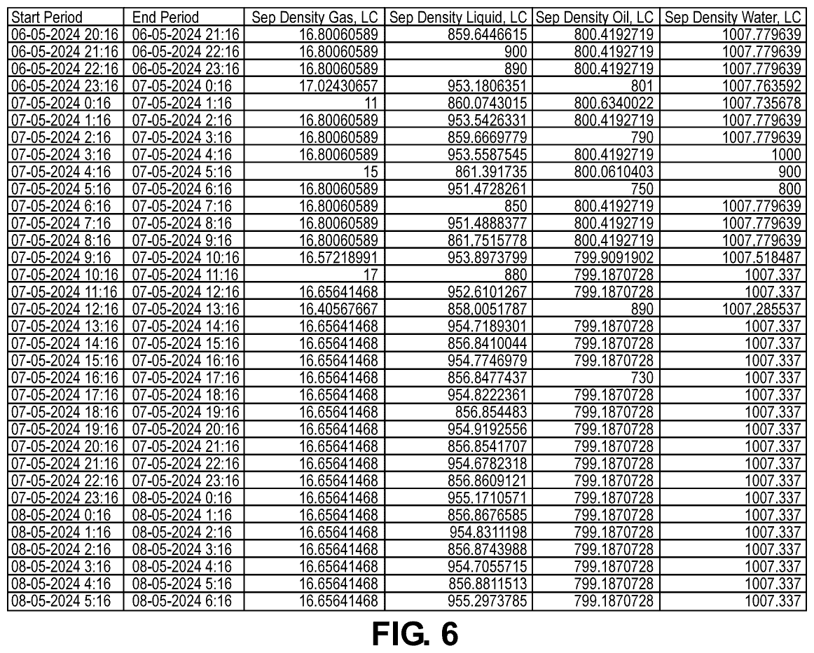

The method 500 may include receiving first data from a separator tank, as at 505. FIG. 6 illustrates a table including a sample of the first data from the separator tank 250, according to an embodiment. The table may contain data for a selected date range and/or within a selected frequency range. The separator tank may be or include the second (e.g., test) separator 250, and it may separate oil, water, and gas from a hydrocarbon fluid that is produced from one or more wellbores 210. The first data may be measured from emissions (e.g., from the gas) that are bled or flared from separator tank 250. The first data may include a plurality of properties of the hydrocarbon fluid. As mentioned above, the properties may include a flow rate of the oil, the water, and the gas; a density of the oil, the water, and the gas; phase fractions of the oil, the water, and the gas; a line temperature of the hydrocarbon fluid; a line pressure of the hydrocarbon fluid; a differential venturi pressure of the hydrocarbon fluid; a water-to-liquid ratio (WLR) of the hydrocarbon fluid; a gas volume fraction (GVF) of the hydrocarbon fluid, or a combination thereof.

The method 500 may also include receiving second data from a flowmeter, as at 510. The flowmeter may be any one or more of the flowmeters 260A-260D. The flowmeter(s) 260A-260D may measure the second data directly from the hydrocarbon fluid. The flowmeter(s) 260A-260D may measure the second data without separating the hydrocarbon fluid (e.g., using the separator tank 250). The flowmeter(s) 260A-260D may measure the second data without bleeding or flaring emissions from the hydrocarbon fluid. The flowmeter(s) 260A-260D may be or include inline, surface, multi-phase flowmeters. The flowmeter(s) 260A-260D may have or include a venturi throat, and the second data may be measured at a single point within the venturi throat. The second data may include the (e.g., same) properties that are measured by the separator tank 250. The first data and the second data may have overlapping date ranges and/or overlapping frequency ranges.

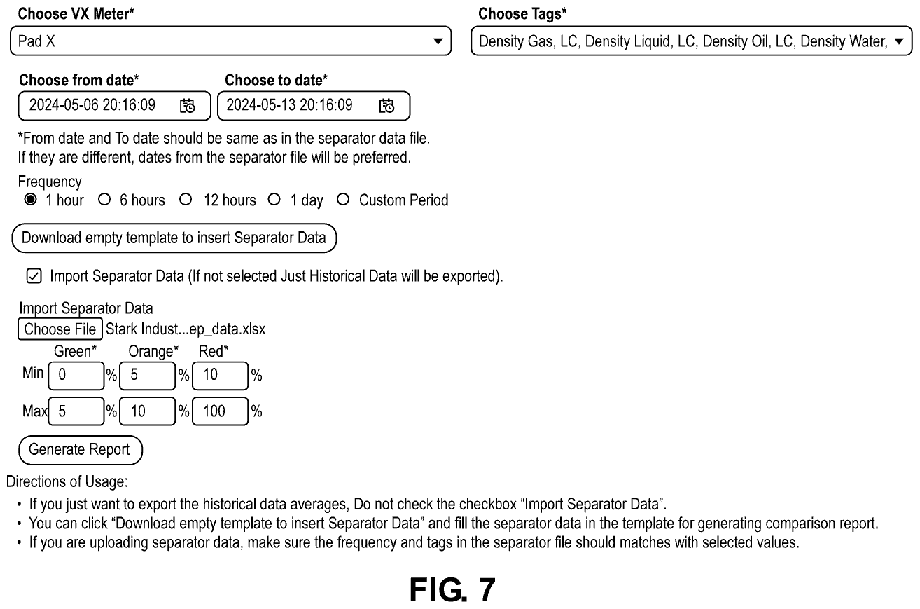

The method 500 may also include comparing the first data and the second data to produce compared data, as at 515. FIG. 7 illustrates a user interface to generate the separator vs flowmeter comparison report, according to an embodiment. A user can select the particular flowmeter (e.g., flowmeter 260A), the tags/channels to be compared, the frequency of data for the comparison, the date range for the comparison, or a combination thereof. As used herein a “tag” and/or “channel” refers to hydrocarbon fluid properties, nuclear material properties, and MPFM configurations such as date, time, and well profile. There may be a provision to download a template that can be filled with the first data (e.g., from the separator tank 250).

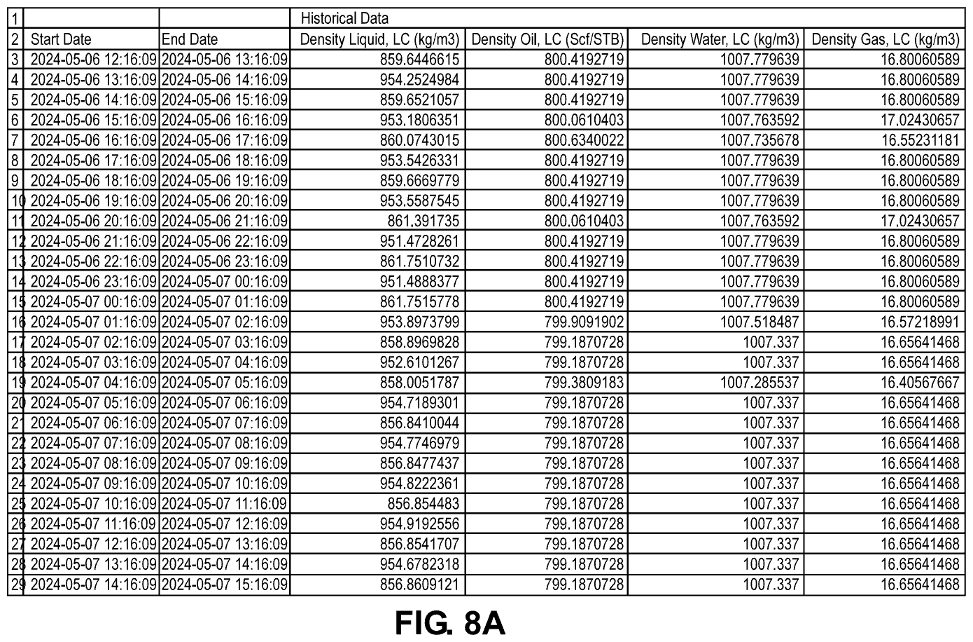

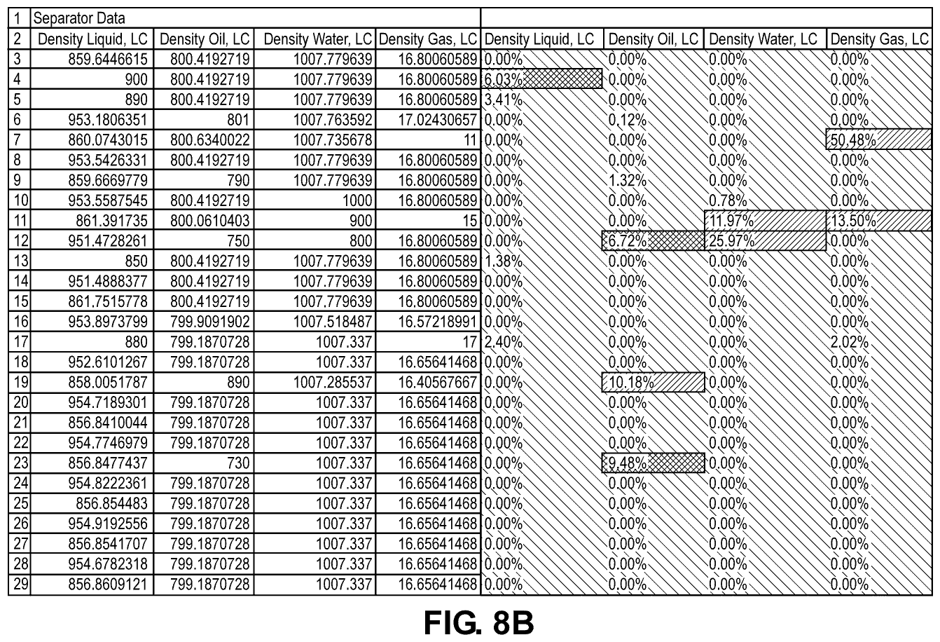

FIGS. 8A and 8B illustrate a comparison report that shows both the first data (e.g., from the separator tank 250) and the second data (e.g., from the flowmeter(s) 260A-260D), according to an embodiment. The differences/deviations are shown in terms of values and percentages.

The method 500 may also include determining one or more differences between the first data and the second data based upon the compared data, as at 520.

The method 500 may also include identifying a first subset of the properties, as at 525. The first subset may be identified in/from differences and/or the comparison report. The first subset may have differences that are less than a first predetermined threshold. The first predetermined threshold may be between 1% and 20%, about 2% and about 10%, or about 3% and about 7%. The first subset of the properties may be identified via highlighting with a first color (e.g., green). This is shown by a first hatching pattern in FIGS. 8A and 8B.

The method 500 may also include identifying a second subset of the properties, as at 530. The second subset may be identified in/from differences and/or the comparison report. The second subset may have differences that are greater than the first predetermined threshold and less than a second predetermined threshold. The second predetermined threshold may be between about 3% and about 30%, about 6% and about 20%, or about 8% and about 12%. The second subset of the properties may be identified via highlighting with a second color (e.g., yellow). This is shown by a second hatching pattern in FIGS. 8A and 8B.

The method 500 may also include identifying a third subset of the properties, as at 535. The third subset may be identified in/from differences and/or the comparison report. The third subset may have differences that are greater than the second predetermined threshold. The third subset of the properties may be identified via highlighting with a third color (e.g., red). This is shown by a third hatching pattern in FIGS. 8A and 8B.

The method 500 may also include generating one or more graphs to show the differences, as at 540. In one embodiment, each of the one or more graphs shows the differences between one of the properties in the first data and the second data. In another embodiment, the one or more graphs show the differences for a plurality of different wellbores 210.

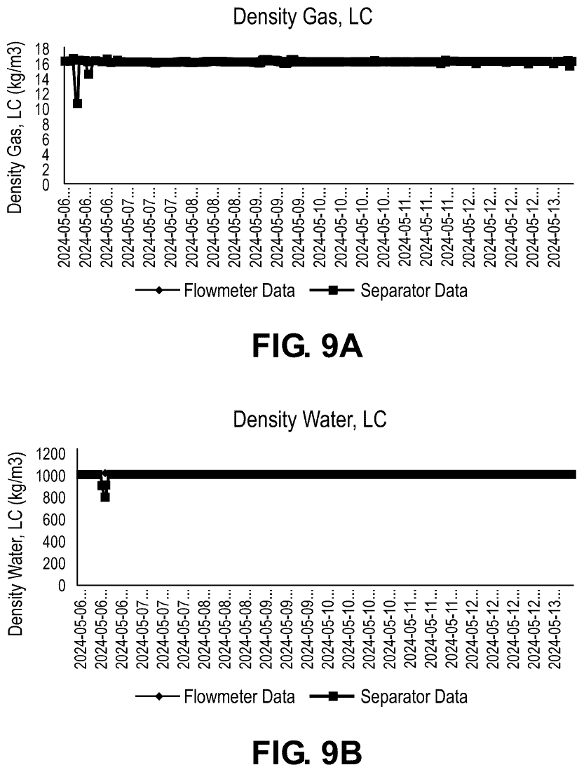

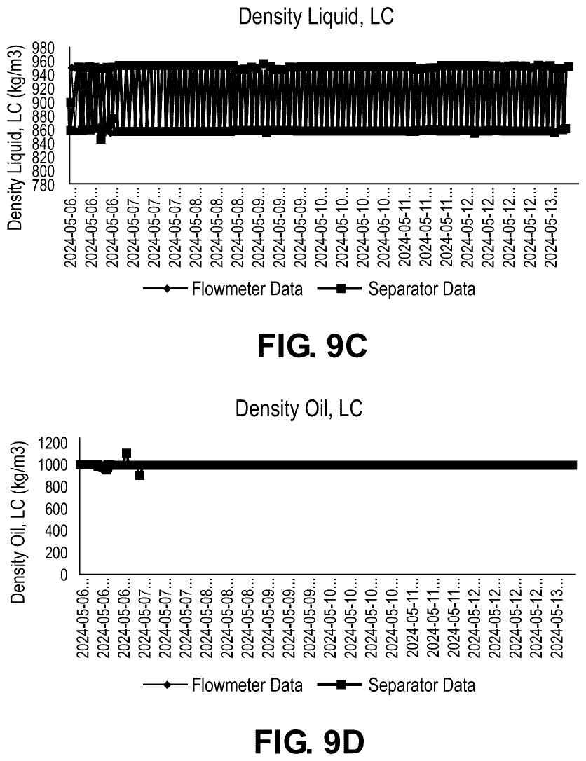

FIGS. 9A-9D illustrate graphs showing trends that may be identified based upon the differences (e.g., in the comparison report), according to an embodiment. More particularly, FIG. 9A illustrates a comparison of the gas density in the first data and the second data, FIG. 9B illustrates a comparison of the liquid density in the first data and the second data, FIG. 9C illustrates a comparison of the oil density in the first data and the second data, and FIG. 9D illustrates a comparison of the water density in the first data and the second data.

The method 500 may also include performing a wellsite action, as at 545. The wellsite action may be in response to the differences, the comparison report, the graphs, or a combination thereof. For example, the wellsite action may be in response to the differences being greater than the first predetermined threshold and/or the second predetermined threshold. Performing the wellsite action may include generating or transmitting a signal that instructs or causes a physical action to occur. The physical action may include calibrating or re-calibrating the flowmeter(s) 260A-260D, so that future measurements from the flowmeter(s) 260A-260D have lesser differences when compared with future measurements from the separator tank 250. Once the differences are minimized (e.g., such that the properties are within the first subset, and no properties are within the second and/or third subset), then the separator tank 250 may be taken offline to reduce or eliminate the emissions. In another embodiment, the physical action may be or include selecting where to drill a wellbore, drilling the wellbore, varying a weight and/or torque on a drill bit that is drilling the wellbore, varying a drilling trajectory of the wellbore, varying a concentration and/or flow rate of a fluid pumped into the wellbore, or the like.

In another embodiment, if there is a difference in the table or the graph(s) that is greater than a predetermined threshold, this may indicate that, at that particular point in time, some action was formed on the flowmeter 260A-260D. For example, flowmeter configurations may be changed, or the flowmeter 260A-260D may be undergoing maintenance.

### Exemplary Computing System

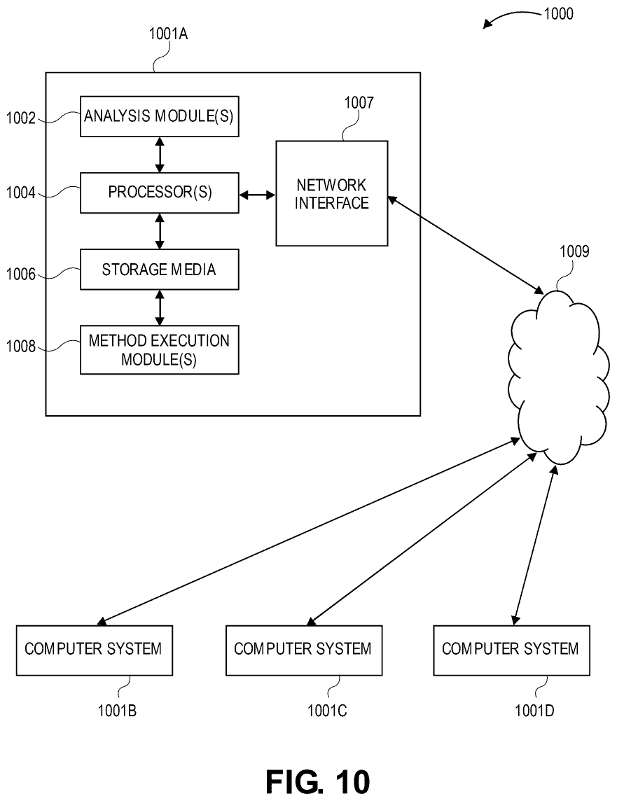

In some embodiments, the methods of the present disclosure may be executed by a computing system. FIG. 10 illustrates an example of such a computing system 1000, in accordance with some embodiments. The computing system 1000 may include a computer or computer system 1001A, which may be an individual computer system 1001A or an arrangement of distributed computer systems. The computer system 1001A includes one or more analysis modules 1002 that are configured to perform various tasks according to some embodiments, such as one or more methods disclosed herein. To perform these various tasks, the analysis module 1002 executes independently, or in coordination with, one or more processors 1004, which is (or are) connected to one or more storage media 1006. The processor(s) 1004 is (or are) also connected to a network interface 1007 to allow the computer system 1001A to communicate over a data network 1009 with one or more additional computer systems and/or computing systems, such as 1001B, 1001C, and/or 1001D (note that computer systems 1001B, 1001C and/or 1001D may or may not share the same architecture as computer system 1001A, and may be located in different physical locations, e.g., computer systems 1001A and 1001B may be located in a processing facility, while in communication with one or more computer systems such as 1001C and/or 1001D that are located in one or more data centers, and/or located in varying countries on different continents).

A processor may include a microprocessor, microcontroller, processor module or subsystem, programmable integrated circuit, programmable gate array, or another control or computing device.

The storage media 1006 may be implemented as one or more computer-readable or machine-readable storage media. Note that while in the example embodiment of FIG. 10 storage media 1006 is depicted as within computer system 1001A, in some embodiments, storage media 1006 may be distributed within and/or across multiple internal and/or external enclosures of computing system 1001A and/or additional computing systems. Storage media 1006 may include one or more different forms of memory including semiconductor memory devices such as dynamic or static random access memories (DRAMs or SRAMs), erasable and programmable read-only memories (EPROMs), electrically erasable and programmable read-only memories (EEPROMs) and flash memories, magnetic disks such as fixed, floppy and removable disks, other magnetic media including tape, optical media such as compact disks (CDs) or digital video disks (DVDs), BLURAY® disks, or other types of optical storage, or other types of storage devices. Note that the instructions discussed above may be provided on one computer-readable or machine-readable storage medium, or may be provided on multiple computer-readable or machine-readable storage media distributed in a large system having possibly plural nodes. Such computer-readable or machine-readable storage medium or media is (are) considered to be part of an article (or article of manufacture). An article or article of manufacture may refer to any manufactured single component or multiple components. The storage medium or media may be located either in the machine running the machine-readable instructions, or located at a remote site from which machine-readable instructions may be downloaded over a network for execution.

In some embodiments, computing system 1000 contains one or more method execution module(s) 1008. In the example of computing system 1000, computer system 1001A includes the method execution module 1008. In some embodiments, a single method execution module may be used to perform some aspects of one or more embodiments of the methods disclosed herein. In other embodiments, a plurality of method execution modules may be used to perform some aspects of methods herein.

It should be appreciated that computing system 1000 is merely one example of a computing system, and that computing system 1000 may have more or fewer components than shown, may combine additional components not depicted in the example embodiment of FIG. 10, and/or computing system 1000 may have a different configuration or arrangement of the components depicted in FIG. 10. The various components shown in FIG. 10 may be implemented in hardware, software, or a combination of both hardware and software, including one or more signal processing and/or application specific integrated circuits.

Further, the steps in the processing methods described herein may be implemented by running one or more functional modules in information processing apparatus such as general purpose processors or application specific chips, such as ASICs, FPGAs, PLDs, or other appropriate devices. These modules, combinations of these modules, and/or their combination with general hardware are included within the scope of the present disclosure.

Computational interpretations, models, and/or other interpretation aids may be refined in an iterative fashion; this concept is applicable to the methods discussed herein. This may include use of feedback loops executed on an algorithmic basis, such as at a computing device (e.g., computing system 1000, FIG. 10), and/or through manual control by a user who may make determinations regarding whether a given step, action, template, model, or set of curves has become sufficiently accurate for the evaluation of the subsurface three-dimensional geologic formation under consideration.

The foregoing description, for purpose of explanation, has been described with reference to specific embodiments. However, the illustrative discussions above are not intended to be exhaustive or limiting to the precise forms disclosed. Many modifications and variations are possible in view of the above teachings. Moreover, the order in which the elements of the methods described herein are illustrated and described may be re-arranged, and/or two or more elements may occur simultaneously. The embodiments were chosen and described in order to best explain the principles of the disclosure and its practical applications, to thereby enable others skilled in the art to best utilize the disclosed embodiments and various embodiments with various modifications as are suited to the particular use contemplated.

What is claimed is:

1. A method for measuring properties of a fluid produced from a wellbore, the method comprising:
receiving first data from a separator tank;
receiving second data from a flowmeter;
comparing the first data and the second data to produce compared data; and
determining differences between the first data and the second data based upon the compared data.

2. The method of claim 1, wherein the separator tank separates gas from the fluid that is produced from the wellbore, and wherein the first data is measured from emissions from the gas that are bled or flared from separator tank.

3. The method of claim 2, wherein the first data and the second data each comprise a plurality of properties of the fluid, and wherein the properties comprise:
a flow rate of the gas;
a density of the gas;
phase fractions of the gas;
a line temperature of the fluid;
a line pressure of the fluid;
a differential venturi pressure of the fluid;
a water-to-liquid ratio (WLR) of the fluid;
a gas volume fraction (GVF) of the fluid; or
a combination thereof.

4. The method of claim 2, wherein the flowmeter measures the second data directly from the fluid.

5. The method of claim 2, wherein the flowmeter measures the second data without separating the fluid with using the separator tank.

6. The method of claim 2, wherein the flowmeter measures the second data without bleeding or flaring emissions from the fluid.

7. The method of claim 1, wherein the flowmeter comprises an inline, surface, multi-phase flowmeter, wherein the flowmeter comprises a venturi throat, and wherein the second data is measured at a single point within the venturi throat.

8. The method of claim 1, wherein the first data and the second data comprise overlapping date ranges and overlapping frequency ranges.

9. The method of claim 1, further comprising generating one or more graphs to show the differences.

10. The method of claim 1, further comprising calibrating the flowmeter to cause the second data to more closely match the first data in response to one or more of the differences being greater than a predetermined threshold.

11. A computing system, comprising:
one or more processors; and
a memory system comprising one or more non-transitory computer-readable media storing instructions that, when executed by at least one of the one or more processors, cause the computing system to perform operations, the operations comprising:
receiving first data from a separator tank, wherein the separator tank separates gas from a hydrocarbon fluid that is produced from a wellbore, wherein the first data is measured from emissions from the gas that are bled or flared from separator tank, wherein the first data comprises a plurality of properties of the hydrocarbon fluid, and wherein the properties comprise:
a flow rate of the oil, the water, and the gas;
a density of the oil, the water, and the gas;
phase fractions of the oil, the water, and the gas;
a line temperature of the hydrocarbon fluid;
a line pressure of the hydrocarbon fluid;
a differential venturi pressure of the hydrocarbon fluid;
a water-to-liquid ratio (WLR) of the hydrocarbon fluid;
a gas volume fraction (GVF) of the hydrocarbon fluid; or
a combination thereof;
receiving second data from a flowmeter, wherein the flowmeter measures the second data directly from the hydrocarbon fluid, wherein the flowmeter measures the second data without separating the hydrocarbon fluid with using the separator tank, wherein the flowmeter measures the second data without bleeding or flaring emissions from the hydrocarbon fluid, wherein the flowmeter comprises an inline, surface, multi-phase flowmeter, wherein the flowmeter comprises a venturi throat, wherein the second data is measured at a single point within the venturi throat, wherein the second data comprises the properties, and wherein the first data and the second data comprise overlapping date ranges and overlapping frequency ranges;
comparing the first data and the second data to produce compared data;
determining differences between the first data and the second data based upon the compared data; and
generating one or more graphs to show the differences, wherein each of the one or more graphs shows the differences between one of the properties in the first data and the second data, and wherein the one or more graphs show the differences for a plurality of different wellbores.

12. The computing system of claim 11, wherein the operations further comprise identifying a first subset of the properties where the differences are less than a first predetermined threshold, wherein the first predetermined threshold is between about 3% and about 7%, and wherein the first subset of the properties are identified via highlighting with a first color.

13. The computing system of claim 12, wherein the operations further comprise identifying a second subset of the properties where the differences are greater than the first predetermined threshold and less than a second predetermined threshold, wherein the second predetermined threshold is between about 8% and about 12%, and wherein the second subset of the properties are identified via highlighting with a second color

14. The computing system of claim 13, wherein the operations further comprise identifying a third subset of the properties where the differences are greater than the second predetermined threshold, and wherein the third subset of the properties are identified via highlighting with a third color

15. The computing system of claim 11, wherein the operations further comprise performing a wellsite action in response to the differences.

16. A non-transitory computer-readable medium storing instructions that, when executed by one or more processors of a computing system, cause the computing system to perform operations, the operations comprising:
receiving first data from a separator tank, wherein the separator tank separates oil, water, and gas from a hydrocarbon fluid that is produced from a wellbore, wherein the first data is measured from emissions from the gas that are bled or flared from separator tank, wherein the first data comprises a plurality of properties of the hydrocarbon fluid, and wherein the properties comprise:
a flow rate of the oil, the water, and the gas;
a density of the oil, the water, and the gas;
phase fractions of the oil, the water, and the gas;
a line temperature of the hydrocarbon fluid;
a line pressure of the hydrocarbon fluid;
a differential venturi pressure of the hydrocarbon fluid;
a water-to-liquid ratio (WLR) of the hydrocarbon fluid; and
a gas volume fraction (GVF) of the hydrocarbon fluid;
receiving second data from a flowmeter, wherein the flowmeter measures the second data directly from the hydrocarbon fluid, wherein the flowmeter measures the second data without separating the hydrocarbon fluid using the separator tank, wherein the flowmeter measures the second data without bleeding or flaring emissions from the hydrocarbon fluid, wherein the flowmeter comprises an inline, surface, multi-phase flowmeter, wherein the flowmeter comprises a venturi throat, wherein the second data is measured at a single point within the venturi throat, wherein the second data comprises the properties, and wherein the first data and the second data comprise overlapping date ranges and overlapping frequency ranges;
comparing the first data and the second data to produce compared data;
determining differences between the first data and the second data based upon the compared data;
generating one or more graphs to show the differences, wherein each of the one or more graphs shows the differences between one of the properties in the first data and the second data, and wherein the one or more graphs show the differences for a plurality of different wellbores;
identifying a first subset of the properties where the differences are less than a first predetermined threshold, wherein the first predetermined threshold is between about 3% and about 7%, and wherein the first subset of the properties are identified via highlighting with a first color;
identifying a second subset of the properties where the differences are greater than the first predetermined threshold and less than a second predetermined threshold, wherein the second predetermined threshold is between about 8% and about 12%, and wherein the second subset of the properties are identified via highlighting with a second color; and
identifying a third subset of the properties where the differences are greater than the second predetermined threshold, wherein the third subset of the properties are identified via highlighting with a third color.

17. The non-transitory computer-readable medium of claim 16, wherein the operations further comprise performing a wellsite action in response to the differences.

18. The non-transitory computer-readable medium of claim 16, wherein the wellsite action is also performed in response to the second subset of the properties or the third subset of the properties.

19. The non-transitory computer-readable medium of claim 18, wherein performing the wellsite action comprises generating or transmitting a signal that instructs or causes a physical action to occur.

20. The non-transitory computer-readable medium of claim 19, wherein the physical action comprises re-calibrating the flowmeter.
